(19) United States
(12) Patent Application Publication
(10) Pub. No.: US 2026/0250131 A1
(43) Pub. Date: Aug. 27, 2026

(54) METHOD FOR PRODUCING A COMPOUND AND APPARATUS FOR PRODUCING A COMPOUND

(71) Applicant: Helander Holding Oy, Turku (FI)

(72) Inventor: Sami TUOMINEN, Turku (FI)

(73) Assignee: Helander Holding Oy, Turku (FI)

(21) Appl. No.: 19/162,190

(22) PCT Filed: Mar. 5, 2024
(86) PCT No.: PCT/FI2024/050085

§ 371 Date: Sep. 4, 2025

(30) Foreign Application Priority Data
Mar. 6, 2023 (FI) 20235258

(51) Int. Cl. C01B 25/165

(52) U.S. Cl. CPC C01B 25/165

(57) ABSTRACT

Page 12 A method for producing a compound comprising at least one of hydrogen or oxygen comprises providing water and a first substance, producing a mixture comprising the water and bubbles comprising the first substance, decreasing a diameter of bubbles comprising the first substance, decomposing a part of the water, and composing a compound at least from the decomposed water and the first substance, and the compound comprising at least one of hydrogen or oxygen. An apparatus is configured for producing a compound comprising at least one of hydrogen or oxygen.

### FIELD OF THE INVENTION

The present invention relates to a method for producing a compound and more particularly to a method according to the preamble of claim 1.

The present invention further relates to apparatus for producing a compound and more particularly to apparatus according to the preamble of claim 11.

### BACKGROUND OF THE INVENTION

In the prior art is known to produce compounds comprising at least one of hydrogen and oxygen with processes so that the processes comprise several reaction steps.

One of the problems associated with the prior art is that producing compounds comprising at least one of hydrogen and oxygen is inefficient as several process steps are needed.

### BRIEF DESCRIPTION OF THE INVENTION

An object of the present invention is to provide a method for producing a compound and apparatus for producing a compound so as to solve or at least alleviate the prior art disadvantages.

The objects of the invention are achieved by a method which is characterized by what is stated in the independent claim 1. The objects of the invention are achieved by apparatus which is characterized by what is stated in the independent claim 11.

The preferred embodiments of the invention are disclosed in the dependent claims.

The invention is based on the idea of providing a method for producing a compound comprising at least one of hydrogen or oxygen. The method comprises providing water and a first substance, producing a mixture comprising the water and bubbles comprising the first substance, decreasing diameter of the bubbles comprising the first substance, decomposing at least one of the first substance and a part of the water to obtain an intermediate, and composing a compound from the intermediate, the compound comprising at least one of hydrogen and oxygen.

According to the present invention the step of decomposing at least one of the first substance and a part of the water to obtain an intermediate comprises decomposing a part of the water or decomposing a part of the water and the first substance to obtain an intermediate.

According to the present invention producing bubbles comprising the first substance and a second substance, decreasing diameter of bubbles comprising the first substance and the second substance, and obtaining intermediate comprising the first substance and the decomposed water.

According to the present invention producing bubbles comprising the first substance, decreasing diameter of bubbles comprising the first substance, decomposing a part of the water and the first substance to obtaining intermediate comprising decomposed the first substance and the decomposed water.

According to the present invention producing bubbles comprising the first substance, decreasing diameter of bubbles comprising the first substance, decomposing a part of the water to obtaining intermediate comprising the first substance and the decomposed water.

According to the present invention producing bubbles comprising the first substance and a second substance, decreasing diameter of bubbles comprising the first substance and the second substance, and decomposing a part of the water, the first substance and the second substance to obtaining intermediate comprising the decomposed water, the decomposed first substance and the decomposed second substance. According to the present invention the method comprises composing a compound at least from the decomposed water and the first substance, and the compound comprising at least one of hydrogen or oxygen.

According to the present invention the method comprises composing a compound at least from the decomposed water and decomposed first substance, and the compound comprising at least one of hydrogen or oxygen.

According to the present invention the first substance being gaseous substance.

According to the present invention the first substance being solid substance, and the method comprises providing a second substance, the second substance being gaseous substance.

According to the present invention the first substance being solid substance.

According to the present invention the first substance comprises particles having diameter less than 180 nm, and the method comprises providing a second substance, and the second substance being gaseous substance.

According to the present invention the first substance comprises particles having diameter less than 180 nm, and the method comprises providing a second substance, the second substance being oxygen.

According to the present invention the first substance comprises solid particles having diameter less than 180 nm, and the method comprises providing a second substance, the second substance being oxygen

Nanobubbles increases an efficiency of the method.

According to the present invention the nano bubble device providing bubbles having a diameter between 50 nm and 180 nm.

It is difficult to produce bubbles having a diameter less than 50 nm.

According to the present invention the first substance being gaseous substance, and the method comprises providing a second substance, the second substance being gaseous substance.

According to the present invention the first substance being gaseous substance, and the method comprises providing a second substance, the second substance being gaseous substance comprising nitrogen.

According to the present invention the first substance being gaseous substance comprising nitrogen.

According to the present invention the first substance being gaseous substance consisting of nitrogen.

According to the present invention the first substance being gaseous substance, and the method comprises providing a second substance, the second substance being nitrogen gas.

According to the present invention the first substance comprises molecules comprising hydrogen atoms.

According to the present invention the first substance comprises molecules comprising hydrogen atoms and carbon atoms.

According to the present invention the first substance comprises molecules consisting of hydrogen atoms and carbon atoms.

According to the present invention the first substance consists of molecules consisting of hydrogen atoms and carbon atoms.

According to the present invention the method comprises obtaining sulphuric acid from the first solid substance, the decomposed water and the second gaseous substance.

The present invention enables producing various compounds, and in the method may be used different substances.

According to the present invention the method comprises producing bubbles comprising the first substance and the second substance, decreasing diameter of bubbles comprising the first substance and the second substance, and composing from the second substance, the decomposed water and the decomposed first substance a compound, the compound comprising the second substance and at least one of hydrogen and oxygen.

The present invention enables producing various compounds, and in the method may be used different substances.

According to the present invention the step of decreasing diameter of the bubbles is carried out with a nano bubble device.

According to the present invention the step of decreasing diameter of the bubbles is carried out with a nano bubble device providing bubbles having diameter less than 180 nm.

According to the present invention the step of decreasing diameter of the bubbles is carried out with a nano bubble device comprising any of the following: pressurized dissolution device, a rotational flow device, a turbulent static mixer, an ejector nozzle and an ultrasonic device and venturi device.

According to the present invention the step of decreasing diameter of the bubbles is carried out with a nano bubble device comprising any of the following: pressurized dissolution device, a rotational flow device, a turbulent static mixer, an ejector nozzle and an ultrasonic device or venturi device, and the nano bubble device providing comprising bubbles having diameter less than 180 nm.

According to the present invention the step of decomposing a part of the water is carried out with electrolytic cell.

The electrolytic cell is an efficient device to carry out decomposing.

According to the present invention the step of decomposing a part of the water and the first substance in the bubbles having decreased size is carried out with electrolytic cell.

According to the present invention the method comprises obtaining compound comprising at least one of hydrogen and oxygen from the step of composing from the first solid substance, the decomposed water and the decomposed first gaseous substance the compound comprising at least one of hydrogen and oxygen.

According to the present invention the method comprises obtaining sulphuric acid from the step of composing a compound from the first solid substance, the decomposed water and the decomposed first gaseous substance.

According to the present invention the method comprises in the step of composing a compound from the decomposed water and from the first substance, the compound comprising acrylonitrile.

According to the present invention the method comprises in the step of composing a compound from the decomposed water and from the first substance, the compound consisting of acrylonitrile.

According to the present invention the method comprises in the step of composing a compound, the compound comprising acrylonitrile and nitric acid.

According to the present invention the method comprises in the step of composing a compound, the compound consisting of acrylonitrile and nitric acid.

Acrylonitrile is widely used to produce polymers.

According to the present invention in the step of composing a compound, the compound comprising ammonia.

According to the present invention in the step of composing a compound, the compound is any one of the following: hypo phosphorous, acrylonitrile, nitric acid and phosphoric acid.

According to the present invention the method comprises obtaining the compound composed from the intermediate, providing water, producing a mixture comprising the water and bubbles comprising the compound composed from the intermediate, decreasing diameter of the bubbles, decomposing at least one of a part of the water and the compound to obtain a second intermediate, and composing a second compound from the second intermediate, and the compound comprising at least one of hydrogen and oxygen.

According to the present invention the step of obtaining the compound composed from the intermediate comprises separating the compound from water, the compound being a solid compound.

According to the present invention the step of obtaining the compound composed from the intermediate, the compound being a gaseous compound.

According to the present invention the method is carried out with any below disclosed embodiment of the apparatus.

The present invention further relates to apparatus for comprising at least one of hydrogen and oxygen. The apparatus comprises a mixing vessel arranged to a receive water and a first substance and the mixing vessel is arranged to produce a mixture comprising the water and bubbles comprising the first substance, a nano bubble device arranged to decrease diameter of bubbles comprising the first substance, and a decomposing device arranged to decompose water and arranged to compose a compound comprising the first substance and at least one of hydrogen and oxygen from the decomposed water.

According to the present invention the mixing vessel comprises a first liquid inlet arranged to receive the water into the mixing vessel, and the mixing vessel comprises a first gas inlet arranged to receive the first substance into the mixing vessel.

According to the present invention the first substance being gaseous substance.

According to the present invention the mixing vessel comprises a first liquid inlet arranged to receive the water into the mixing vessel, and the mixing vessel comprises a first gas inlet arranged to receive the first substance into the mixing vessel, and the mixing vessel comprises the nano bubble device.

According to the present invention the mixing vessel comprises a first liquid inlet arranged to receive the water into the mixing vessel, the mixing vessel comprises a first gas inlet arranged to receive the first substance into the mixing vessel, and the mixing vessel comprises a second gas inlet arranged to receive the second substance into the mixing vessel.

According to the present invention the second substance being gaseous substance.

According to the present invention the mixing vessel comprises a first liquid inlet arranged to receive the water into the mixing vessel, the mixing vessel comprises a first gas inlet arranged to receive the first substance into the mixing vessel, the mixing vessel comprises a second gas inlet arranged to receive the second substance into the mixing vessel, and the mixing vessel comprises the nano bubble device.

According to the present invention the nano bubble device comprising any one of the following: pressurized dissolution device, a rotational flow device, a turbulent static mixer, an ejector nozzle and an ultrasonic device and venturi device.

According to the present invention the electrolytic cell comprises an anode element and a cathode element.

According to the present invention the electrolytic cell comprises an anode element and a cathode element, and the anode element comprises platinum and the cathode element comprises stainless steel.

According to the present invention the electrolytic cell comprises an anode element and a cathode element, and the anode element and the cathode element comprises diamond.

According to the present invention the electrolytic cell comprises an anode element and a cathode element the anode element and the cathode element comprises black diamond.

According to the present invention the decomposing device comprises an anode element and a cathode element.

According to the present invention the decomposing device comprises an anode element and a cathode element, and the anode element comprises platinum and the cathode element comprises stainless steel.

According to the present invention the decomposing device comprises an anode element and a cathode element and the anode element and the cathode element comprises diamond.

According to the present invention the decomposing device comprises an anode element and a cathode element, and the anode element and the cathode element comprises black diamond.

According to the present invention the decomposing device comprises an anode element and a cathode element, the anode element comprises an ion exchange film and the cathode element comprises stainless steel.

According to the present invention the ion exchange film of the anode element may partially homogeneous membrane.

The partially homogenous membrane remains active even it is contact with air.

According to the present invention the ion exchange film of the anode element may be a heterogenous membrane or homogeneous membrane.

Diamond anode does not corrode but diamonds are expensive.

According to the present invention the decomposing device is arranged to receive the mixture comprising gaseous substance and the liquid substance from the nano bubble device.

According to the present invention the electrolytic cell device is arranged to receive the mixture comprising first substance and the liquid substance from the nano bubble device.

According to the present invention the decomposing device may comprises an electrolytic cell.

An advantage of the invention is that compounds can be produced more. efficiently, and furthermore new compounds can be produced.

### BRIEF DESCRIPTION OF THE DRAWINGS

The invention is described in detail by means of specific embodiments with reference to the enclosed drawings, in which,

FIG. 1 shows principles of the method according to the present invention; and

FIG. 2 shows schematically one embodiment of apparatus for producing a compound comprising at least one of hydrogen and oxygen according to the present invention; and

FIG. 3 shows schematically one embodiment of apparatus for producing a compound comprising at least one of hydrogen and oxygen according to the present invention.

### DETAILED DESCRIPTION OF THE INVENTION

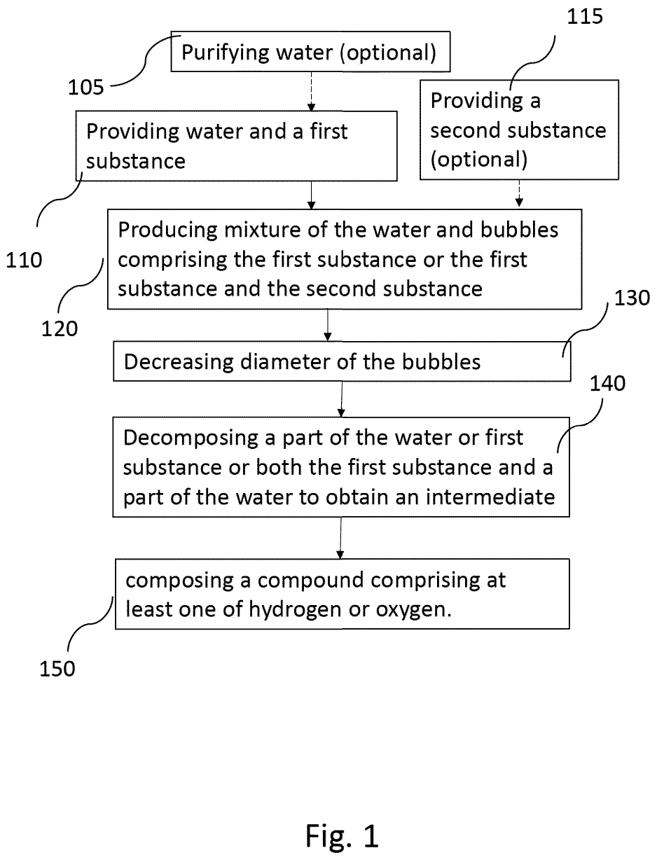

FIG. 1 discloses a method for producing a compound comprising at. least one of hydrogen or oxygen. The method comprises in the step 110 providing water and a first substance. In the step 120, the method comprises producing a mixture comprising the water and bubbles comprising the first substance. In the step 130, the method comprises decreasing diameter of bubbles comprising the first substance. In the step 140, the method comprises decomposing a part of the water. In the step 150, the method comprises composing a compound at least from the decomposed water and the first substance, and the compound comprising at least one of hydrogen or oxygen.

In certain embodiments, the first substance being gaseous substance, and 1-100 wt-% of the first substance is decomposed based on a total weight of the first substance.

In certain embodiments, the first substance being gaseous substance, and 20-90 wt-% of first substance is decomposed based on a total weight of the gaseous substance.

In certain embodiments, the method comprises a step of decomposing 1-99 wt-% or 0,1-50 wt-% or 20-80 wt-% of the water based on the total weight of the water,

In certain embodiments, the first substance being gaseous substance.

In certain embodiments, the first substance being solid substance, and the method comprises providing a second substance, the second substance being gaseous substance.

In certain embodiments, the first substance comprises particles having diameter less than 180 nm, and the method comprises providing a second substance, and the second substance being gaseous substance.

In certain embodiments, the first substance comprises particles having diameter less than 180 nm., and the method comprises providing a second substance, the second substance being oxygen.

In the context of this application, the diameter of particle means greatest straight line segment that passes through the centre of the particle and whose endpoints lie on an outer surface of the particle.

In certain embodiments, the first substance being gaseous substance, and the method comprises providing a second substance, the second substance being gaseous substance.

In certain embodiments, the first substance being gaseous substance, and the method comprises providing a second substance, the second substance being gaseous substance comprising nitrogen.

In certain embodiments, the first substance being gaseous substance, and the method comprises providing a second substance, the second substance being nitrogen gas.

In certain embodiments, the first substance comprises molecules comprising hydrogen atoms.

In certain embodiments, the first substance comprises molecules comprising hydrogen atoms and carbon atoms.

In certain embodiments, the first substance comprises molecules consisting of hydrogen atoms and carbon atoms.

In certain embodiments, the first substance consists of molecules consisting of hydrogen atoms and carbon atoms.

In certain embodiments, producing bubbles comprising the first substance and the second substance, decreasing diameter of bubbles comprising the first substance and the second substance, and composing from the second substance, the decomposed water and the decomposed first substance a compound, the compound comprising the second substance and at least one of hydrogen and oxygen.

In certain embodiments, decreasing diameter of the bubbles is carried out with a nano bubble device 2.

In certain embodiments, decreasing diameter of the bubbles is carried out with a nano bubble device 2 providing bubbles having diameter less than 180 nm.

In certain embodiments, decreasing diameter of the bubbles is carried out with a nano bubble device 2 comprising any of the following: pressurized dissolution device, a rotational flow device, a turbulent static mixer, an ejector nozzle and an ultrasonic device and venturi device.

In certain embodiments, decreasing diameter of the bubbles is carried out with a nano bubble device 2 comprising any of the following: pressurized dissolution device, a rotational flow device, a turbulent static mixer, an ejector nozzle and an ultrasonic device or venturi device, and the nano bubble device 2 providing comprising bubbles having diameter less than 180 nm.

In certain embodiments, the method comprises decomposing a part of the water is carried out with electrolytic cell 35.

In certain embodiments, decomposing a part of the water and the first substance in the bubbles having decreased size is carried out with electrolytic cell 35.

In certain embodiments, the method comprises obtaining compound comprising at least one of hydrogen and oxygen from the step of composing from the first solid substance, the decomposed water and the decomposed first gaseous substance the compound comprising at least one of hydrogen and oxygen.

In certain embodiments, the method comprises obtaining sulphuric acid from the step of composing a compound from the first solid substance, the decomposed water and the decomposed first gaseous substance.

In certain embodiments, in the step of composing a compound from the decomposed water and from the first substance, the compound comprising acrylonitrile.

In certain embodiments, in the step of composing a compound from the decomposed water and from the first substance, the compound consisting of acrylonitrile.

In certain embodiments, the method is carried out with apparatus 100 disclosed below.

### Example 1

In the prior art sulfuric acid (H₂SO₄) is produced, for instance, with the wet sulfuric acid process (WSA process) which comprises the steps of:
Combustion: 2 H₂S+3 O₂ [symbol] 2 H₂O+2 SO₂
Oxidation: 2 SO₂+O₂ [symbol] 2 SO₃ [in the presence of a vanadium (V) oxide catalyst]
Hydration: SO₃+H₂O [symbol] H₂SO₄ (g)

The inventors surprisingly found out that sulfuric acid may be produced more efficiently by combining sulphur in nano bubbles and decomposed water. The water was decomposed with electrolytic cell.

### Example 2

In the prior art, Ammonia (an inorganic compound of nitrogen and hydrogen with the formula NH₃), for instance, may be produced by a process called self-propagating high-temperature synthesis (SHS) where a metal nitride is ignited in a hydrogen atmosphere.

The inventors surprisingly found out that ammonia may be produced more efficiently by combining decomposed water and gaseous nitrogen in nano bubbles. The water was decomposed with electrolytic cell.

### Example 3

Acrylonitrile may be produced, for instance, by catalytic ammoxidation of propylene, also known as the SOHIO process 2CH₃—CH═CH₂+2NH₃+3O₂→2CH₂═CH—C≡N+6H₂O. In the SOHIO process, propylene, ammonia, and air (oxidizer) are passed through a fluidized bed reactor containing the catalyst at 400-510° C. and 50-200 kPa.

The inventors surprisingly found out that acrylonitrile was produced more efficiently when First ammonia was produced according to example 2 and separated from the water and then water was decomposed, and finally decomposed water and ammonia in nano bubbles was combined. Furthermore, nitric acid was composed in the process.

### Example 4

In the prior art phosphoric acid HaPO4 is produced, for instance is produced from apatite and sulfuric acid as Ca₃(PO₄)₂+3H₂SO₄→2H₃PO₄+3CaSO₄.

The inventors surprisingly found out that phosphoric acid may be produced efficiently from decomposed water and white phosphorus P₄ in nano bubbles, Phosphorus pentoxide as an intermediate was formed from the decomposed water and phosphorus in nano bubbles. Then phosphoric acid H₃PO₄ was composed.

### Example 5

First was produced a gaseous composition comprising white phosphorus (P₄) and hydrogen. Then was produced a mixture comprising water and bubbles of white phosphorus and hydrogen. Then diameter of the bubbles was decreased. Then a part of the water was decomposed. Then hypo phosphorous acid H₃PO₂ was composed.

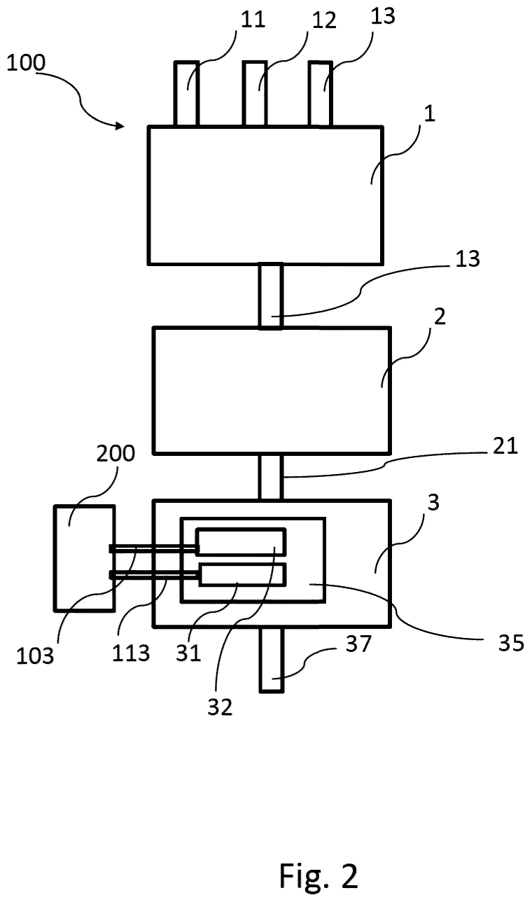

FIG. 2 shows one embodiment of apparatus 100 for producing hydrogen of the present invention.

The apparatus 100 comprises a mixing vessel 1 arranged to a receive water and a first substance and the mixing vessel 1 is arranged to produce a mixture comprising the water and bubbles comprising the first substance, a nano bubble device 2 arranged to decrease diameter of bubbles comprising the first substance, and a decomposing device 3 arranged to decompose water and arranged to compose a compound comprising the first substance and at least one of hydrogen and oxygen from the decomposed water.

In certain embodiments, the mixing vessel 1 being a pressure vessel designed to hold gases or liquids at a pressure between 1-5 atm.

In certain embodiments, the mixing vessel 1 comprises a first liquid inlet 11 arranged to receive the water into the mixing vessel 1, the first substance being gaseous substance, and the mixing vessel 1 comprises a first gas inlet arranged to receive the first substance into the mixing vessel 1.

In certain embodiments, the mixing vessel 1 comprises a first liquid inlet 11 arranged to receive the water into the mixing vessel 1, the first substance being gaseous substance, and the mixing vessel 1 comprises a first gas inlet 12 arranged to receive the first substance into the mixing vessel 1, and the mixing vessel 1 comprises the nano bubble device 2.

In certain embodiments, the mixing vessel 1 comprises a first liquid inlet 11 arranged to receive the water into the mixing vessel 1, the first substance being gaseous substance, the mixing vessel 1 comprises a first gas inlet 12 arranged to receive the first substance into the mixing vessel 1, the second substance being gaseous substance, and the mixing vessel 1 comprises a second gas inlet 13 arranged to receive the second substance into the mixing vessel 1.

In certain embodiments, the mixing vessel 1 comprises a first liquid inlet 11 arranged to receive the water into the mixing vessel 1, the first substance being gaseous substance, the mixing vessel 1 comprises a first gas inlet 12 arranged to receive the first substance into the mixing vessel 1, the second substance being gaseous substance, the mixing vessel 1 comprises a second gas inlet 13 arranged to receive the second substance into the mixing vessel 1, and the mixing vessel 1 Comprises the nano bubble device 2.

In certain embodiments, the nano bubble device 2 comprising any one of the following: pressurized dissolution device, a rotational flow device, a turbulent static mixer, an ejector nozzle and an ultrasonic device and venturi device.

In certain embodiments, the nano bubble device 2 is configured to provide bubbles having a diameter less than 180 nm.

In pressurized dissolution method ultrafine bubble generation is based on the principles of Henry's Law, which relates the concentration of a gas to the partial pressure. This means that more gas can be dissolved into a solution at a higher pressure.

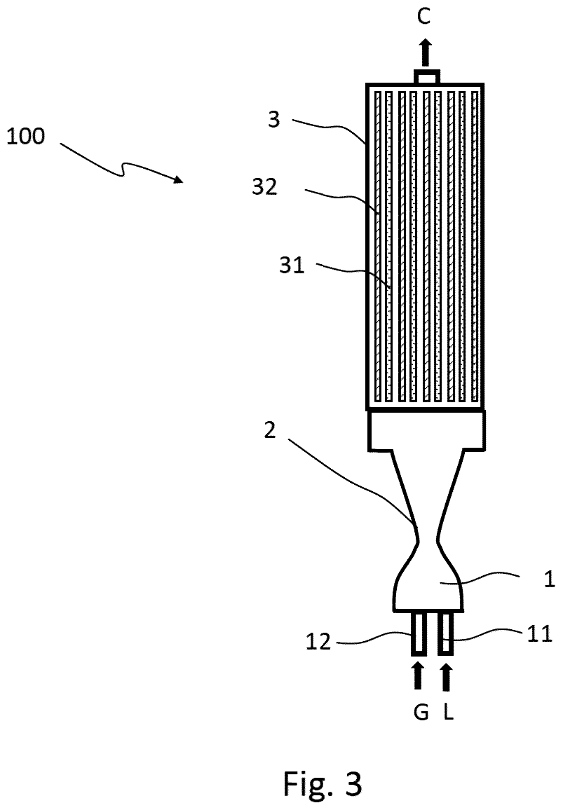

The principle of the ultrafine bubble generator is as follows: Via a venturi system the liquid and the gas are mixed together, in the next step in the mixing box the gas is melted into the water via pressurization. In the last step via a nozzle the water and gas is discharged. Due to drastic drop in pressure of the supersaturated liquid gas solution, the gas is expelled as fine bubbles and ultrafine bubbles in the liquid. The FIG. 3 shows one embodiment of a device of the pressurized dissolution method. Liquid is pumped into the unit under pressure. By narrowing the diameter of the pipe, the speed of the incoming liquid flow is increased, which converts most of the pump pressure into dynamic pressure, thus reducing static pressure and air being suctioned through negative pressure. After the liquid and suctioned gas become saturated with bubbles, the liquid/gas flow is sent through a wider pipe to reduce the speed of the flow, where dynamic pressure is converted back to static pressure and the process of pressurized dissolution of gas takes place. After the gas is completely dissolved into the liquid, the liquid/gas is ejected at once using atmospheric pressure, causing the liquid to become over-saturated, and massive ultra-fine nanobubbles are released.

Rotational flow is also often called Swirl Method or Spiral Flow. This fine bubble generator generates bubbles according to the Bernoulli's principle. In fluid dynamics, Bernoulli's principle states that an increase in the speed of a fluid occurs simultaneously with a decrease in pressure or a decrease in the fluid's potential energy. Centuries later, fine bubble generators are made based on this principle. Followed 50 years later by the Swirling jet flame. In the mid-nineties, the first swirling type micro-bubbles was invented in Japan. The principle of the fine bubble generator is as follows: water is put into a cylindrical tank from the topside and made to flow in a spiral downwards. From the centre bottom of the cylinder, the gas is sucked in. The rotating water is sheared to the top of the cylinder, producing fine bubbles. However, it's generally acknowledged in the ultrafine bubble industry that the bubble concentration of the pressurized dissolution method is higher than the rotational flow.

The static mixer has its origin from mixing two liquids. Instead of mixing two liquids, there is also the possibility of mixing a liquid and a gas. This technology is based on the principle of creating a vortex and bringing into the vortex a gas very effectively. Due to the turbulent flow gas will break the vortex and the collisions between water and gas creates the nanobubbles. The benefits of the static mixers are that they have a relatively simple design, and they can treat large volumes of water at once with relatively little energy compared to many of the other above nanobubble generators. Finally, they are not sensitive to clogging. The acniti Turbiti technology is a combination of the turbulent static mixer and the Ejector Nozzle.

In the ejector nozzle nanobubble generator type, liquid flow channels in the cylindrical generator are designed to shrink and stepwise enlarge. The gas is. brought in under negative pressure at the most reduced pressure point and reduced to a number of nanobubbles by cavitation. In this device, the water flow is highly turbulent, and the gas is reduced to nanobubbles by cavitation. Ejector nozzles are closely related to hydrodynamic cavitation generators, with this method cavitation is generated by the flow of liquid through a simple geometry under controlled conditions. In this nanobubble generator, when the pressure falls below the vapor pressure of the liquid, the liquid flashes, generating a number of cavities. The cavities collapse when the pressure recovers. The collapse of the cavitation bubbles starts some physicochemical effects such as shock waves, shear forces and chemical reactions. Free radicals are sometimes generated by these processes.

The hammermill rotation concept is a unique concept compared to all the other nanobubble generation techniques, as it does not use a pump to generate nanobubble. Instead, it uses a motor with hammers mounted on the shaft. The motor turns at a velocity of 3400 rotations per minute in a tube. The tube fills with water from the top, and the gas injection is also from the top. The hammers on the shaft dissolve the gas and crushes the gas into nanobubble at the bottom of the unit the nanobubbles come out. The hammer rotation concept is the most energy friendly way to generate nanobubbles as it is not moving large amounts of water and doesn't need a high pressure but uses all its energy to crush the gas. The line-up of hammermill rotation nanobubble generators is called the microStar nanobubble generator.

In certain embodiments, the decomposing device 3 comprises an electrolytic cell 35.

In certain embodiments, the electrolytic cell 35 comprises an anode element 31 and a cathode element 32.

In certain embodiments, the electrolytic cell 35 comprises an anode element 31 and a cathode element 32, and the anode element 31 comprises platinum and the cathode element 32 comprises stainless steel.

In certain embodiments, the electrolytic cell 35 comprises an anode element 31 and a cathode element 32, and the anode element 31 and the cathode element 32 comprises diamond.

In certain embodiments, the electrolytic cell 35 comprises an anode element 31 and a cathode element 32, and the anode element 31 and the cathode element 32 comprises black diamond.

In certain embodiments, the electrolytic cell 35 is arranged to receive the mixture comprising first substance and the liquid substance from the nano bubble device 2.

In certain embodiments, the decomposing device 3 comprises an electrolytic cell 35 comprising an anode element 31 and a cathode element 32.

In certain embodiments, the anode element 31 comprises platinum and the cathode element 32 comprises stainless steel.

In certain embodiments, the anode element 31 and the cathode element 32 comprises diamond.

In certain embodiments, the anode element 31 and the cathode element 32 comprises black diamond.

In certain embodiments, the anode element 31 comprises an ion exchange film and the cathode element 32 comprises stainless steel.

In certain embodiments, the decomposing device 3 is arranged to receive the mixture from the nano bubble device 2.

In certain embodiments the apparatus 100 comprises a gas outlet 37 arranged to enable obtaining a gaseous compound from the apparatus 100.

In certain embodiments the apparatus 100 comprises a first passage 13 arranged to transfer a mixture comprising the water and bubbles from the mixing vessel 1 to the nano bubble device 2.

In certain embodiments the apparatus 100 comprises a second passage 21 arranged to transfer a mixture comprising the water and the nano bubbles from the nano bubble device 2 to the decomposing device 3.

FIG. 3 shows one embodiment of apparatus 100 for producing a compound of the present invention.

In this embodiment, the mixing vessel 1 comprises the nano bubble device 2.

It should be noted that the embodiment shown in FIG. 3 may comprise features of embodiments shown in FIG. 2.

The invention has been described above with reference to the examples shown in the figures. However, the invention is in no way restricted to the above examples but may vary within the scope of the claims.

1. A method for producing a compound comprising at least one of hydrogen and oxygen, the method comprising:
providing water and a first substance,
producing a mixture comprising the water and bubbles comprising the first substance,
decreasing a diameter of the bubbles comprising the first substance,
decomposing at least one of the first substance and a part of the water to obtain an intermediate, and
composing a compound from the intermediate, the compound comprising at least one of hydrogen and oxygen.

2. The method according to claim 1, wherein:
the first substance is a solid substance, and the method comprises providing a second substance, the second substance being a gaseous substance; or
the first substance comprises particles having diameter less than 180 nm[[., and the method comprises providing a second substance, and the second substance being a gaseous substance; or
the first substance comprises particles having a diameter less than 180 nm, and the method comprises providing a second substance, the second substance being oxygen.

3. The method according to claim 1, wherein:
the first substance is a gaseous substance; or
the first substance is gaseous substance, and the method comprises providing a second substance, the second substance being a gaseous substance; or
the first substance is a gaseous substance comprising nitrogen; or
the first substance is a gaseous substance consisting of nitrogen.

4. The method according to claim 1, wherein:
the first substance comprises molecules comprising hydrogen atoms; or
the first substance comprises molecules comprising hydrogen atoms and carbon atoms; or
the first substance comprises molecules consisting of hydrogen atoms and carbon atoms; or
the first substance consists of molecules consisting of hydrogen atoms and carbon atoms.

5. The method according to claim 1, comprising:
producing bubbles comprising the first substance and a second substance, decreasing diameter of bubbles comprising the first substance and the second substance, and obtaining intermediate comprising the first substance and the decomposed water; or
producing bubbles comprising the first substance, decreasing diameter of bubbles comprising the first substance, decomposing a part of the water and the first substance to obtaining intermediate comprising decomposed the first substance and the decomposed water; or
producing bubbles comprising the first substance, decreasing diameter of bubbles comprising the first substance, decomposing a part of the water to obtaining intermediate comprising the first substance and the decomposed water; or
producing bubbles comprising the first substance and a second substance, decreasing diameter of bubbles comprising the first substance and the second substance, and decomposing a part of the water, the first substance and the second substance to obtaining intermediate comprising the decomposed water, the decomposed first substance and the decomposed second substance.

6. The A method according to claim 1, wherein:
the decreasing of the diameter of the bubbles is carried out with a nano bubble device; or
the decreasing of the diameter of the bubbles is carried out with a nano bubble device providing bubbles having diameter less than 180 nm; or
the decreasing of the diameter of the bubbles is carried out with a nano bubble device comprising any of the following: a pressurized dissolution device, a rotational flow device, a turbulent static mixer, an ejector nozzle and an ultrasonic device and venturi device; or
the step of decreasing diameter of the bubbles is carried out with a nano bubble device comprising any of the following: a pressurized dissolution device, a rotational flow device, a turbulent static mixer, an ejector nozzle and an ultrasonic device or venturi device, and the nano bubble device providing comprising bubbles having diameter less than 180 nm.

7. The A method according to claim 1, wherein:
the decomposing of the part of the water is carried out with an electrolytic cell; or
the decomposing of the part of the water and the first substance in the bubbles having decreased size is carried out with the electrolytic cell.

8. The method according to claim 1, wherein:
the first substance is sulphur, and in the composing of the compound, the compound is sulphuric acid.

9. The method according to claim 1, wherein:
in the composing of the compound, the compound comprises acrylonitrile; or
in the composing of the compound, the compound consists of acrylonitrile; or
in the composing of the compound, the compound comprises acrylonitrile and nitric acid; or
in the composing of the compound, the compound consists of acrylonitrile and nitric acid; or
in the composing of the compound, the compound comprises ammonia; or
in the composing of the compound, the compound is any one of the following; hypo phosphorous, acrylonitrile, nitric acid and phosphoric acid.

10. The method according to claim 1, wherein:
the method is carried out with an apparatus comprising:
a mixing vessel arranged to a receive water and a first substance, the mixing vessel being arranged to produce a mixture comprising the water and bubbles comprising the first substance,
a nano bubble device arranged to decrease diameter of bubbles comprising the first substance, and
a decomposing device arranged to decompose water and arranged to compose a compound comprising the first substance and at least one of hydrogen and oxygen from the decomposed water.

11. An apparatus for producing a compound comprising at least one of hydrogen and oxygen, the apparatus comprising:
a mixing vessel arranged to a receive water and a first substance, the mixing vessel being arranged to produce a mixture comprising the water and bubbles comprising the first substance,
a nano bubble device arranged to decrease diameter of bubbles comprising the first substance, and
a decomposing device arranged to decompose water and arranged to compose a compound comprising the first substance and at least one of hydrogen and oxygen from the decomposed water.

12. The apparatus according to claim 11, wherein:
the mixing vessel comprises a first liquid inlet arranged to receive the water into the mixing vessel, and the mixing vessel comprises a first gas inlet arranged to receive the first substance into the mixing; or
the mixing vessel comprises a first liquid inlet arranged to receive the water into the mixing vessel, and the mixing vessel comprises a first gas inlet arranged to receive the first substance into the mixing vessel, and the mixing vessel comprises the nano bubble device; or
the mixing vessel comprises a first liquid inlet arranged to receive the water into the mixing vessel, the mixing vessel comprises a first gas inlet arranged to receive the first substance into the mixing vessel, and the mixing vessel comprises a second gas inlet arranged to receive the second substance into the mixing vessel; or
the mixing vessel comprises a first liquid inlet arranged to receive the water into the mixing vessel, the mixing vessel comprises a first gas inlet arranged to receive the first substance into the mixing vessel, the mixing vessel comprises a second gas inlet arranged to receive the second substance into the mixing vessel, and the mixing vessel comprises the nano bubble device.

13. The apparatus according to claim 11, wherein
the nano bubble device comprises any one of the following: a pressurized dissolution device, a rotational flow device, a turbulent static mixer, an ejector nozzle and an ultrasonic device and venturi device.

14. The apparatus according to claim 11, wherein an electrolytic cell device comprises:
an anode element and a cathode element; or
an anode element and a cathode element, and the anode element comprises platinum and the cathode element comprises stainless steel; or
an anode element and a cathode element, and the anode element and the cathode element comprises diamond; or
an anode element and a cathode element, and the anode element and the cathode element comprises black diamond.

15. The apparatus according to claim 11, wherein:
the electrolytic cell device is arranged to receive the mixture comprising first substance and the liquid substance from the nano bubble device.
